(19) United States
(12) Patent Application Publication
(10) Pub. No.: US 2023/0181150 A1
(43) Pub. Date: Jun. 15, 2023

(54) NON-INVASIVE MONITORING OF INTRACRANIAL HEMORRHAGE

(71) Applicant: Infrascan Inc., Philadelphia, PA (US)

(72) Inventors: Meltem Izzetoglu, Bryn Mawr, PA (US); Baruch Ben-Dor, Radnor, PA (US); Hasan Ayaz, Bryn Mawr, PA (US)

(21) Appl. No.: 18/057,105

(22) Filed: Nov. 18, 2022

Related U.S. Application Data
(60) Provisional application No. 63/287,815, filed on Dec. 9, 2021.

(51) Int. Cl. A61B 8/08; A61B 5/03

(52) U.S. Cl. CPC A61B 8/0808; A61B 5/031; A61B 5/4064

(57) ABSTRACT

A method of assessing intracranial hemorrhage in a subject may include at a first time point: measuring, for a plurality of times, optical density (OD) value at first location on a first side of a head of the subject at a first time point, and a first corresponding location on a second side of the head of the subject. Change in optical density (ΔOD) is computed by subtracting each OD value measured on the first side from each corresponding OD value measured on the second side. A predetermined number of largest absolute ΔOD values are eliminated from the ΔOD values and a first average ΔOD value is computed by averaging the remainder of ΔOD values. The process is repeated at a second time point to obtain a second average ΔOD. Progression of intracranial hemorrhage is determined based on a difference between the first average ΔOD and the second average ΔOD.

### CROSS-REFERENCE TO RELATED APPLICATIONS

This application claims to benefit of priority to U.S. Provisional Pat. Application No. 63/287,815, filed on Dec. 9, 2021, which is incorporated by reference herein by its entirety for all purposes.

### FIELD OF THE DISCLOSURE

The present disclosure relates generally to the field of non-invasive detection of brain trauma, and in particular to non-invasive monitoring of brain bleeds.

### BACKGROUND

A traumatic injury to the head such as, from a fall, motor vehicle collision or an assault can cause damage to blood vessels that run along the surface of the brain. Such damage can cause intracranial bleeding (hemorrhage) and/or accumulation (hematoma) of blood outside the blood vessels. Detection and monitoring of intracranial hematomas and/or hemorrhage is critical in triage and treatment of patients with head trauma.

One of the techniques used for detection of brain bleeds uses near infrared spectroscopy to assess the presence of a brain hematoma in a patient. The basic method for hematoma detection is based on the differential light absorption of the injured vs. the non-injured part of brain. Under normal circumstances, the brain’s absorption should be symmetrical when comparing left and right sides. When additional underlying extra vascular blood is present, due to internal bleeding, there is a greater local concentration of hemoglobin and consequently the absorbance of the light is greater while the reflected component is commensurately less. This differential is detectable via light sources and detectors placed on symmetrical lobes of the skull. U.S. Pat. No. 8,060,189 entitled System and Method for Detection of Brain Hematoma, which is hereby incorporated by reference as if set forth in its entirety herein, describes systems and methods for detection of brain hematoma using near infrared spectroscopy.

In many cases, head trauma injuries are time critical, and thus, the ability to diagnose them at the scene of the injury using portable equipment is desirable. Moreover, if intracranial hemorrhage is detected at the scene of injury where transportation to a medical facility for surgical intervention is not immediately possible, having the ability monitor the hemorrhage is desirable to enable triage procedures and prevent further brain damage caused by a growing hematoma resulting from the hemorrhage, thereby improving patient outcome and reducing recovery time.

### SUMMARY

The embodiments disclosed herein are proposed to enable non-invasive monitoring of hemorrhage and a size of a hematoma using, for example, near infrared spectroscopy measurements. The methods and systems of the present disclosure are derived from the realization that variability in measured optical signals caused, for example, by hair on the patient’s head, make it difficult to use near infrared spectroscopy for monitoring intracranial hematoma and/or hemorrhage over a period of time.

Advantageously, the systems and methods described herein reduce variability in near infrared spectroscopy signals measured at different time points by eliminating a predetermined number of largest absolute values of change in optical density so as to remove outlier measurements caused by measurement artifacts such as, for example, the patient’s hair.

Accordingly, in at least one embodiment, a method of assessing intracranial hemorrhage in a subject may include: (a) at a first time point: (aa) measuring, for a plurality of times, optical density (OD) value at first location on a first side of a head of the subject at a first time point, and a first corresponding location on a second side of the head of the subject, (ab) computing a set of values of a change (ΔOD) in optical density by subtracting each OD value measured on the first side from each of the OD values measured on the second side, (ac) eliminating a predetermined number of largest absolute Δ0D values from the set of Δ0D values, and (ad) computing a first average ΔOD value by averaging remaining of Δ0D values from among the set of Δ0D values after step (ac). The method further includes: (b) at second time point, repeating steps (aa)-(ad) obtain a second average Δ0D value; and (c) determining a progression of intracranial hemorrhage based on a difference between the first average ΔOD and the second average ΔOD.

In an aspect of the present application, non-transitory machine-readable medium storing instructions to cause one or more processors to perform operations including: (a) at a first time point: (aa) measuring, for a plurality of times, optical density (OD) value at first location on a first side of a head of the subject at a first time point, and a first corresponding location on a second side of the head of the subject, (ab) computing a set of values of a change (ΔOD) in optical density by subtracting each OD value measured on the first side from each of the OD values measured on the second side, (ac) eliminating a predetermined number of largest absolute Δ0D values from the set of Δ0D values, and (ad) computing a first average ΔOD value by averaging remaining of Δ0D values from among the set of Δ0D values after step (ac). The operations further include: (b) at second time point, repeating steps (aa)-(ad) obtain a second average Δ0D value; and (c) determining a progression of intracranial hemorrhage based on a difference between the first average ΔOD and the second average ΔOD.

In accordance with at least one embodiment, a system for assessing intracranial hemorrhage in a subject may include an optical probe, a memory device to store instructions and one or more processors operably connected to the optical probe and configured to execute the instructions stored on the memory device. The optical probe is configured to measure optical density from a portion of a subject’s head. The instructions cause the one or more processors to: (a) at a first time point: (aa) cause the optical probe to measure, for a plurality of times, optical density (OD) value at first location on a first side of a head of the subject at a first time point, and a first corresponding location on a second side of the head of the subject, (ab) compute a set of values of a change (ΔOD) in optical density by subtracting each OD value measured on the first side from each of the OD values measured on the second side, (ac) eliminate a predetermined number of largest absolute Δ0D values from the set of Δ0D values, and (ad) compute a first average ΔOD value by averaging remaining of Δ0D values from among the set of Δ0D values after step (ac). The instructions further cause the one or more processors to: (b) at second time point, repeat steps (aa)-(ad) obtain a second average Δ0D value; and (c) determine a progression of intracranial hemorrhage based on a difference between the first average ΔOD and the second average ΔOD.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the present disclosure are described below with reference to the drawings. The illustrated embodiments are intended to illustrate, but not to limit, the present disclosure. The drawings contain the following figures:

FIG. 1A is a schematic view of a diagnostic system shown during use on a patient in accordance with an exemplary embodiment of the present disclosure.

FIG. 1B is a schematic view of a diagnostic system shown during use on a patient in accordance with an exemplary embodiment of the present disclosure.

FIG. 2 is a block diagram of an optical probe used with embodiments of the present disclosure.

FIG. 3 is a schematic of an embodiment of the present disclosure utilizing remote analysis.

FIG. 4 is a graph of Δ0D measurements of four brain lobes of a patient over 8 consecutive hourly scans.

FIG. 5A is a graph of standard deviation of ΔOD as calculated using different averaging algorithms from measurements in a group of subjects having hematoma.

FIG. 5B is a graph of standard deviation of ΔOD as calculated using different averaging algorithms from measurements in a group of subjects having no hematoma.

FIG. 6A is a graph of average of ΔOD as calculated using different averaging algorithms from measurements in a group of subjects having hematoma.

FIG. 6B is a graph of average of ΔOD as calculated using different averaging algorithms from measurements in a group of subjects having no hematoma.

FIGS. 7A-7C show graphs demonstrating the reduction in signal variability and increase in smoothness of the data by increasing the number of outliers eliminated during the data processing.

FIG. 8 is a graph showing distribution of change measurements obtained in accordance with an embodiment of the present disclosure in subjects with no hematoma, subjects with hematoma and healthy volunteers.

FIG. 9 illustrates how small shifts in measurement location can add to signal variability in measurements of small hematomas.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein.

Embodiments of the present disclosure relate to non-invasive monitoring of hemorrhage and a size of a hematoma using, for example, near infrared spectroscopy measurements. Advantageously, the methods and systems disclosed herein reduce the variability in near infrared spectroscopy measurements to enable more accurate non-invasive monitoring of intracranial hematoma and/or hemorrhage over a period of time.

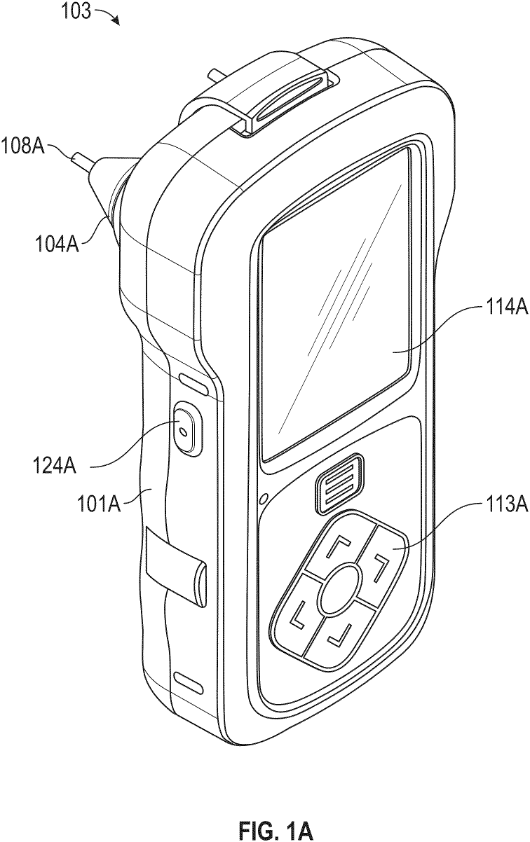

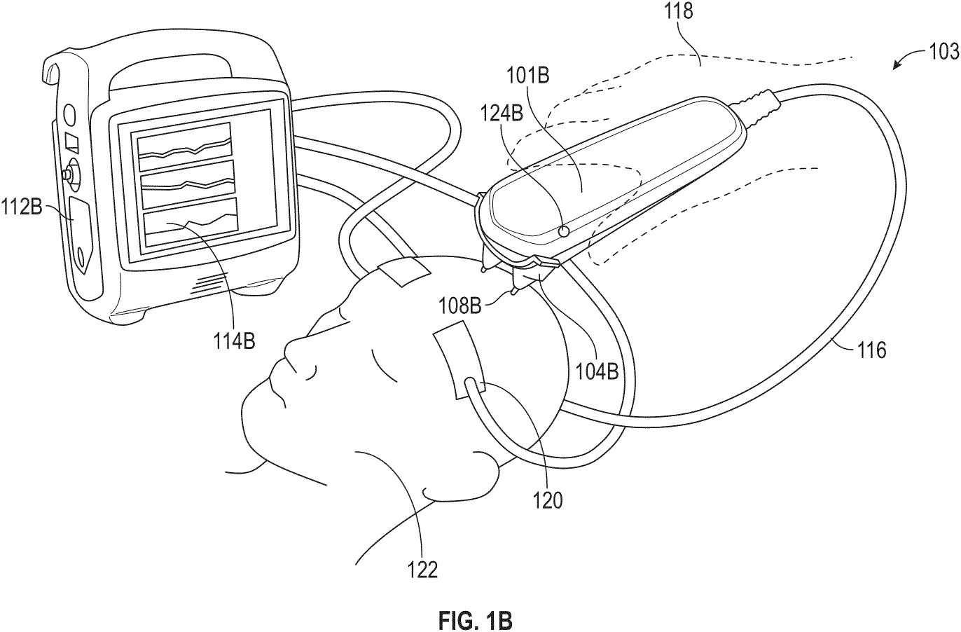

Examples of near infrared spectroscopy instruments on which the methods and systems of the present disclosure can be implemented can be found in U.S. Pat. Publication No. 2020/0229712, which is incorporated herein by reference in its entirety. In at least some embodiments, the methods disclosed herein can be implemented on a diagnostic system illustrated in FIG. 1.

FIGS. 1A and 1B show examples of a diagnostic system 103 in use on a head 122 of a patient. The diagnostic system 101 may include a probe 101A or probe 101B. While probe 101A and probe 101B illustrate two different form factors for the probe 101, both designs 101A and 101B are configured to function in essentially the same way. Throughout the specification, the probes 101A and 101B are collectively referred to as probe 101.

The probe 101 (i.e., 101A or 101B) is configured to be grasped and operated by a single hand 118 of the user. The probe 101 includes a plurality of optical fibers 108 (i.e., 108A/108B, respectively for probes 101A/101B) that extend from the probe and are applied to a patient’s head 122.

The system 103 includes an electronic monitoring device 112 which may include a user interface 114 to enable a user to visualize the measurements obtained by the system and/or interact with the system. Additionally, the probes 101A and 101B may respectively include button 124A and 124B for initiating a measurement.

Referring to FIGS. 1A and 1B the probe 101A/101B is configured and sized to be grasped and operated by a single hand. The probe 101A/101B includes a plurality of optical elements 108A/108B that extend from the probe and are applied to a patient’s head. Probe 101A/101B may have a dual light detector configuration, or a dual light source configuration.

In some embodiments, as illustrated in FIG. 1A, a user interface 114A and a control panel 113A may be included in the body of (i.e., integrated with) the probe 101A.

In some embodiments, as illustrated in FIG. 1B, the user interface 114B may be provided separately from the probe 101B as part of an electronic monitoring device 112B. In such embodiments, the probe 101B may be connected wirelessly or via a cable 116 to an electronic monitoring device 112B. In some embodiments, cable 116 may additionally function to provide power and/or light to one or more electronic components within probe 101B.

As shown in FIG. 1B, a user may use his/her hand 118 to place the optical fibers 108B extending from the probe 101B at a desired location on the head 122 of a patient. Preferably the optical fibers 108 extend between the patient’s hair to directly contact the user’s scalp.

The user may then perform a measurement by activating a control on the user interface 114 (i.e., user interface 114A/114B respectively for probes 101A/101B). In some embodiments, the user interface 114 may include a touch screen, such as a capacitive or resistive touch screen. During use, an operator may attempt to clean any blood from the head in the measurement area, to reduce its impact on the light measurements.

Thus, disclosed embodiments may provide a multifunction diagnostic system. The system may serve as a diagnosis, resuscitation, and surgery aid for traumatic brain injury and hemorrhagic shock patients using near-infrared spectroscopy (NIRS) technology. A current problem in trauma care, particularly for out-of-hospital trauma, is the lack of methods and systems to identify, monitor, and trend physiologic (biochemical, metabolic or cellular) parameters. Noninvasive devices to detect brain and body hemorrhage, edema, blood and tissue oxygen, and assess vital organ perfusion and cognitive function are desperately needed. Such technology provides critical baselines for monitoring and assessment of trauma victims during resuscitation efforts and en route during evacuation. Disclosed embodiments may perform multiple monitoring and diagnostic functions in far forward field conditions.

Disclosed embodiments may further include additional sensors coupled to the electronic monitoring device, such as a sensor 120. In some embodiments, several different NIRS sensors 120 are placed on the head, torso, and/or on the limbs of the patient. Disclosed embodiments may enable multiple measurements in an integrated multifunction device, providing considerable weight and volume savings since many of the needed system elements are mutual (computer, screen, batteries, etc.).

In some embodiments, system 103 may perform multiple functions, including, but not limited to, full head scan for brain hematoma diagnosis (using probe 101), full head scan for local cerebral oximetry measurement (using probe 101), bilateral forehead cerebral oximetry/hypoxia monitoring (using sensor(s) 120), local tissue oximetry monitoring in extremities (using sensors similar to sensor(s) 120, placed on an extremity, like a leg or an arm), heart rate and heart rate variability ((using sensor(s) 120), respiration rate ((using sensor(s) 120), bilateral forehead cerebral edema monitoring ((using sensor(s) 120), and/or sedation monitoring in field surgery ((using sensor(s) 120).

The system 103 may help to avoid or at least reduce a patient’s exposure to radiation by reducing the need for computed tomography (CT or CAT) scans to diagnose the patient with a brain trauma. Reducing the patient’s exposure to radiation, in some embodiments, may have particular benefits in use with children or pregnant women where the exposure to radiation may be more detrimental as compared to non-pregnant adults. In some embodiments, the system 103 is configured for pediatric use.

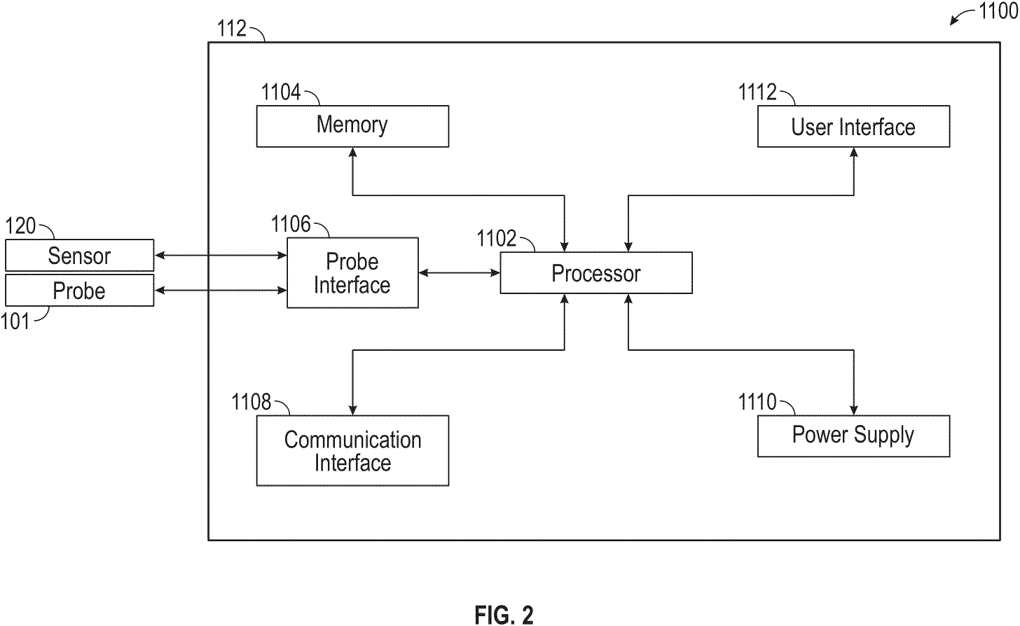

FIG. 2 shows a block diagram 1100 of a device used with embodiments of the present invention. A processor 1102 is coupled to various components, including memory 1104. Memory 1104 may include a non-transitory computer readable medium such as random-access memory (RAM), static random-access memory (SRAM), read-only memory (ROM), flash, magnetic storage, optical storage, and/or other suitable storage technology. The memory 1104 may contain machine instructions, that when executed by processor 1102, perform steps in accordance with embodiments of the present invention. Processor 1102 may include one or more cores. Note that while one processor 1102 is shown in FIG. 2, in some embodiments, multiple processors may be used. The processors can include microprocessors, microcontrollers, digital signal processors (DSPs) and/or other suitable processors.

Block diagram 1100 further includes input/output (I/O) or probe interface 1106. The probe interface 1106 may include one or more pins configured to generate and/or receive signals from peripheral devices such as probe 101A/101B (FIG. 1A, FIG. 1B) and/or sensor 120 (FIG. 1B).

Block diagram 1100 may further include communication interface 1108. Communication interface 1108 may include a wired and/or wireless Ethernet interface, a serial port, a USB (Universal Serial Bus) port, or other suitable mechanism for transmitting and receiving data and/or configuration information. Communication interface 1108 may include a cellular transceiver, near field communication (NFC) transceiver, Bluetooth™ transceiver, or other suitable transceiver to enable wireless communication. In embodiments, the processor 1102 communicates with remote computing devices via the Internet, by way of communication interface 1108. In some embodiments, the processor may transmit raw data, such as light intensity readings and measurement locations to a remote computing device for analysis. The remote computing device may then perform an analysis and transmit results back to the processor 1102 for rendering on user interface 1112. In this way, computation-intensive operations can be performed on a remote device, reducing the computing and power requirements of the portable device (1000) used for in situ measurements.

User interface 1112 may include a screen or a touch screen such as a capacitive or resistive touch screen. User interface 1112 may include a keyboard, mouse or other suitable pointing device, joystick, one or more buttons, or other suitable mechanism to enable control of the probe 101A/101B (FIGS. 1A, 1B).

Block diagram 1100 further includes power supply 1110. Power supply 1110 may include an AC (alternating current power supply), DC (direct current power supply), battery, or other suitable power source for providing power, enabling the portability of probe 101A/101B.

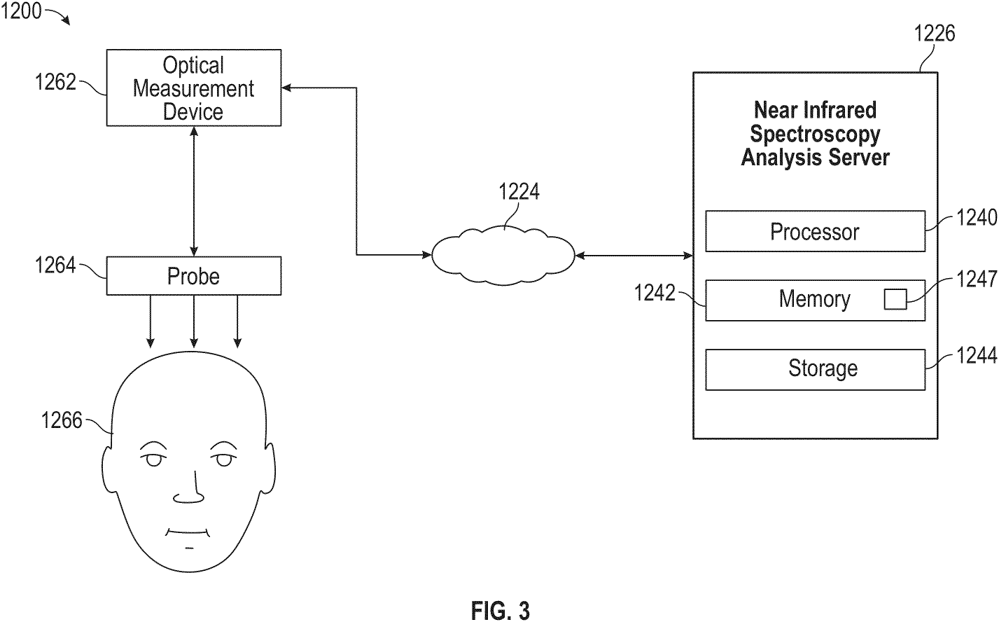

FIG. 3 shows an embodiment 1200 of the present disclosure utilizing remote analysis. Embodiment 1200 includes an optical measurement device 1262, which may be similar to probe 101A/101B of FIGS. 1A, 1B and contain components such as those indicated in block diagram 1100 of FIG. 2. Probe 1264, which may be similar to probe 101A/101B of FIGS. 1A, 1B is applied to a patient 1266 at a variety of locations for taking measurements of light intensity.

In some embodiments, the probe 1264 may be in communication with measurement device 1262 via a wired connection such as shown in FIG. 1B with cable 116. The wired connection may include multiple conduits for electrical and/or optical signals to travel to and from the probe and the optical measurement device. The optical measurement device, using a communication interface such as shown as 1108 in FIG. 2, communicates to a near infrared spectroscopy analysis server 1226 via network 1224. In embodiments, network 1224 may be the Internet, a wide area network (WAN), a local area network (LAN), or any other suitable network.

Oximetry analysis server 1226 may comprise processor 1240, memory 1242, and storage 1244. Instructions 1247 for executing embodiments of the present invention are shown stored in memory 1242. In some embodiments, the oximetry analysis server 1226 may perform an analysis of raw data acquired by probe 1264. The oximetry analysis server 1226 may then send results back to the optical measurement device 1262 and/or other electronic devices to report the results. In some embodiments the oximetry analysis server 1226 may be implemented in a cloud computing environment. In some embodiments, the oximetry analysis server 1226 may be implemented as a virtual machine operating in a cloud computing environment.

The embodiment 1200 depicted in FIG. 3 enables enhanced communication of results. In some embodiments, results of the oximetry measurements may be automatically sent via e-mail, text message, or other suitable mechanism to one or more persons on a distribution list, such as physicians and/or nurses, to quickly disseminate the trauma analysis information. In such embodiments, the optical measurement device 1262 may still perform some analysis locally, in the event that the optical measurement device is used in a situation where network 1224 is unavailable. In this way, the optical measurement device 1262 can operate in an offline mode, and still provide some trauma analysis results to the operator of the optical measurement device 1262.

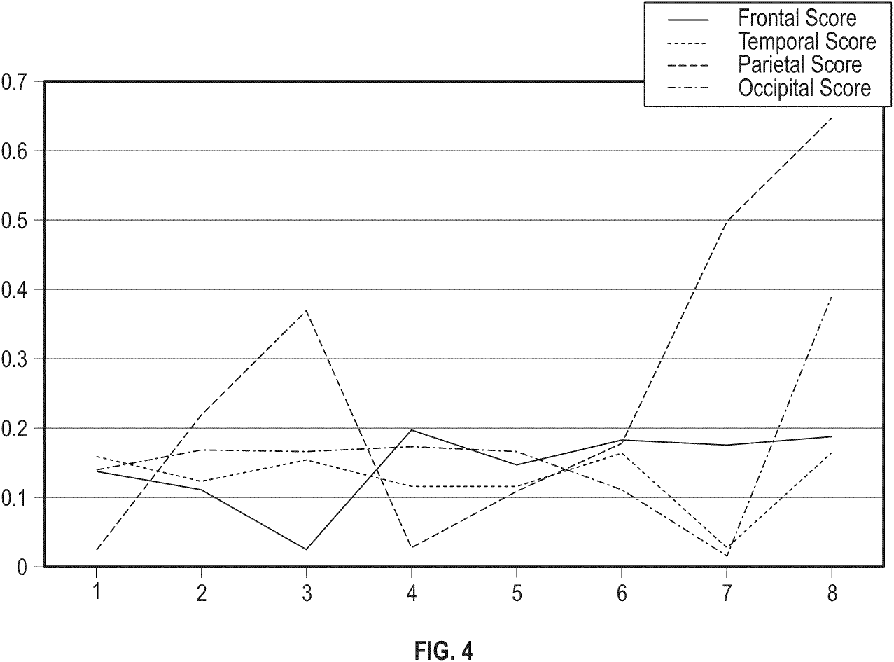

The measured Δ0D of several first patients in a trial showed very large variability over time in patients that according to a CT scan were stable as seen in the graphs shown in FIG. 4, thereby highlighting some of the issues in monitoring the progress (or lack thereof) of hemorrhage or hematoma in patients over a period of time. It was found that the large variability in the measured signal may render the measurements unusable.

Accordingly, the present disclosure provides scanning sequence and data processing algorithms that can alleviate these issues in detecting, monitoring and triage of intracranial hematomas and/or hemorrhages. The presently disclosed methods and systems accomplish this by improving signal stability and reducing variability in ΔOD measured over a period of time among different patients. The presently disclosed methods and systems, thus, stabilize the measurements by: (a) making more measurements at each location, regardless of Δ0D value, and (b) applying a suitable averaging algorithm to smooth signal variability.

To that end, data of patients that were stable on CT was reviewed and the measurements were grouped to create a simulation of repeat measurements at each location. For this analysis, only the data from patients that had 7-9 repeated measurements was used. Only the first measurements were used (repeated measurements of suspected hematomas were ignored). The data was arranged into 3 groups of 3 measurements (for 7 and 8 measurements, some data was used in two groups).

All head locations without CT confirmed hematoma were grouped into No Hematoma locations and all hematoma locations were grouped separately.

All STD: standard deviation of original 7-9 consecutive ΔOD measurements was calculated. The standard deviation serves as an indicator for signal stability.

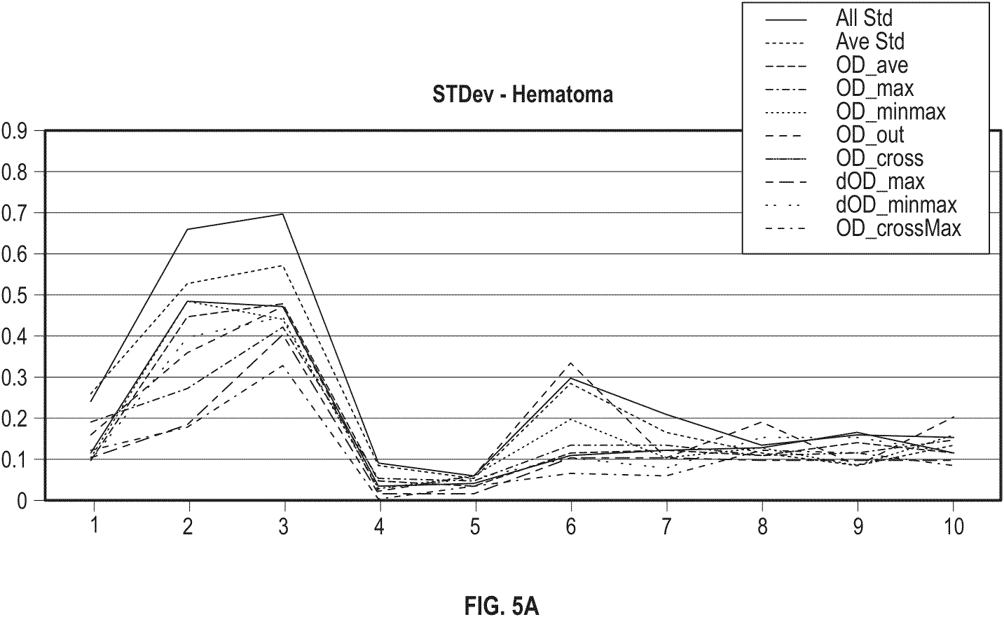

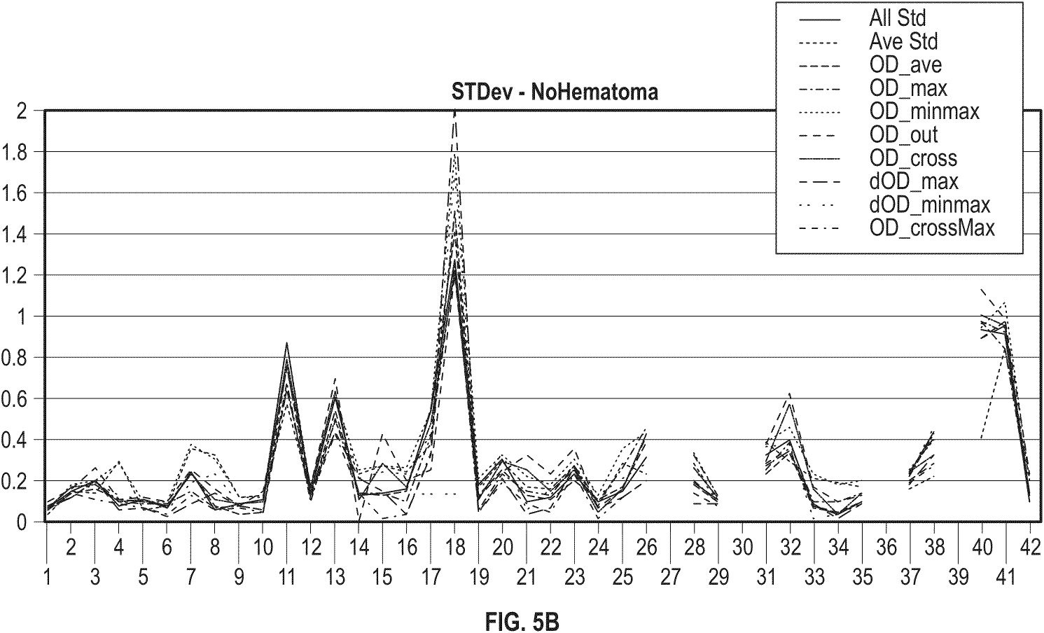

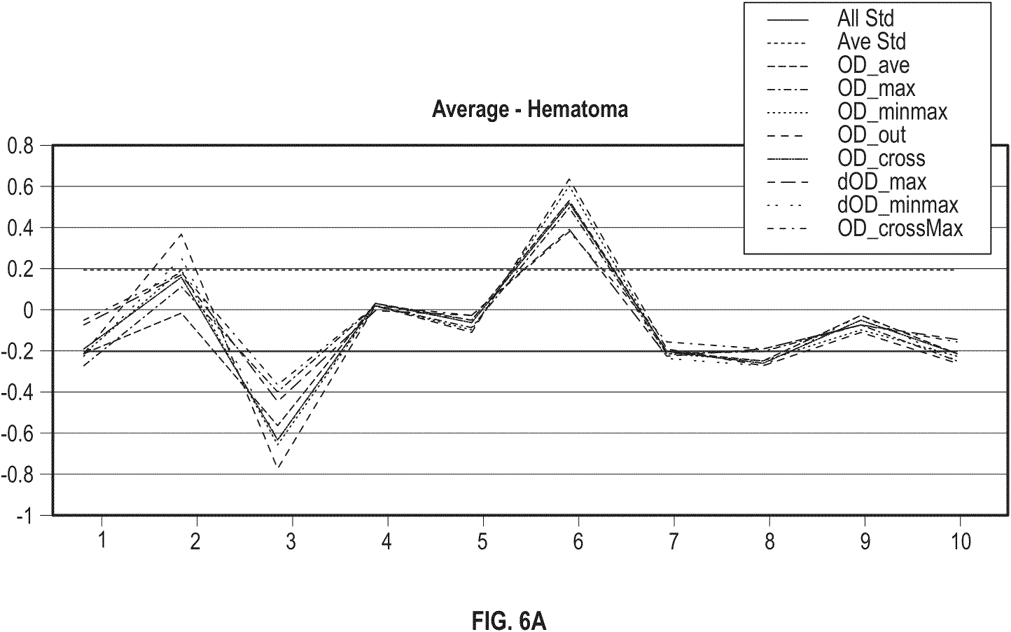

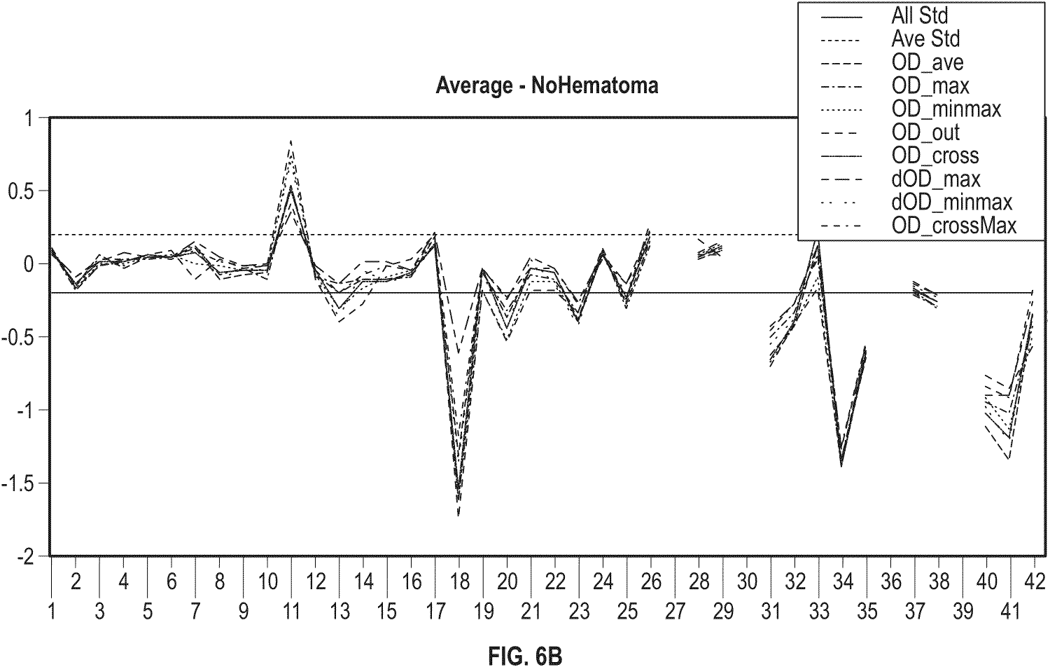

Nine different signal processing algorithms as detailed below were applied and their results compared (FIGS. 5A, 5B, 6A, 6B):

1: Ave STD: Calculate 3 ΔOD pairs in each group, calculate standard deviation in each group and then average them.

2: OD_Ave: Calculate average of 3 left and right ODs in each group, calculate Δ0D and then calculate standard deviation.

3: OD_Max: Eliminate the maximum OD on the left and on the right and average the remaining two on each side, calculate Δ0D and then calculate standard deviation.

4: OD_MinMax: Eliminate the minimum and the maximum OD on the left and on the right, calculate Δ0D and then calculate standard deviation.

5: OD_out: Eliminate the statistical outlier OD on the left and on the right and average the remaining two or three on each side, calculate Δ0D and then calculate standard deviation.

6: OD_Cross: Calculate 9 ΔOD pairs in each group crossing each OD on the right and the 3 left ODs, eliminate the statistical outlier ΔOD, average the rest and then calculate standard deviation.

7: ΔOD_Max: Calculate 3 ΔOD pairs in each group, eliminate the maximum ΔOD, average the rest and then calculate standard deviation.

8: ΔOD_MinMax: Calculate 3 ΔOD pairs in each group, eliminate the minimum and maximum Δ0D and then calculate standard deviation.

9: OD_CrossMax: Calculate 9 ΔOD pairs in each group crossing the 3 right ODs and the 3 left ODs, eliminate the 3 maximum absolute ΔODs, average the rest of the original ODs and then calculate standard deviation.

In determining the suitable algorithm, the following criteria were used:
For no-hematoma and healthy volunteers’ cases the average ΔOD should be as close to zero as possible and to have the lowest standard deviation.
For the hematoma cases the algorithm should yield the lowest standard deviation.

Based both on the processes disclosed herein, and prior data it the most suitable algorithm for stabilization of output was determined to be algorithm 9 (OD_CrossMax). Algorithm 7 (Δ0D_Max) was also found to be suitable and met the above-discussed criteria.

Without wishing to be bound by theory, algorithms 9 and 7 may work well for the following reasons: hair trapped under the fibers of the scanning apparatus is one of the main sources of signal variability. The trapped hair result in a higher OD and hence higher Δ0D values. Thus, eliminating maximal Δ0D values is likely to remove hair effects and reduce signal variability. Algorithm 7 (AOD_Max) removes the max OD value, but may also remove a potentially good measurement on the contralateral side. In contrast, algorithm 9 (OD_CrossMax) removes the max OD value, while still using the potentially good measurement on the contralateral side.

Thus, while any of the data processing algorithms disclosed herein may be used when monitoring intracranial hematoma and/or hemorrhage for a given patient over a period of time, at least some embodiments of the present disclosure utilize algorithm 9 (OD_CrossMax) disclosed herein for monitoring intracranial hematoma and/or hemorrhage in a patient over a period of time.

In accordance with at least some embodiments, the process of monitoring intracranial hematoma and/or hemorrhage may include scanning a head of a patient using any of the optical density measurement systems disclosed herein.

In an aspect of the present disclosure, the process for assessing intracranial hemorrhage and/or hematoma in a subject may include: (a) at a first time point: (aa) measuring, for a plurality of times, optical density (OD) value at first location on a first side of a head of the subject at a first time point, and a first corresponding location on a second side of the head of the subject, (ab) computing a set of values of a change (ΔOD) in optical density by subtracting each OD value measured on the first side from each of the OD values measured on the second side, (ac) eliminating a predetermined number of largest absolute Δ0D values from the set of Δ0D values, and (ad) computing a first average ΔOD value by averaging remaining of Δ0D values from among the set of Δ0D values after step (ac). Next, (b) at second time point, steps (aa)-(ad) are repeated to obtain a second average Δ0D value. At (c) a progression of intracranial hemorrhage is determined based on a difference between the first average ΔOD and the second average ΔOD.

In some embodiments, the optical density at (aa) is measured three times, and the predetermined number in (ac) is three. In some embodiments, the method may further include repeating (a)-(c) at a second location on the first side and a second corresponding location on the second side of the head of the subject.

In some embodiments, the second side may be a contralateral side of the head of the subject. In such embodiments, the first and second corresponding locations on the second side refer to the same location on the contralateral side of the head as the first and second locations on the first side. Thus, for example, if the first location on the first side corresponds to the frontal lobe on the right side, the first location on the second, i.e., contralateral, side corresponds to the frontal lobe on the left side.

Thus, in at least one embodiment, the process includes the scanning may be performed in the following scanning sequence:

Left frontal - 3 repeat measurements. The first measurement is used with calibration phase first and the later 2 measurements are using the parameters from that first calibration.

Right frontal - 3 repeat measurements. All 3 measurements are using the parameters from the left measurement first calibration.

Left temporal - 3 repeat measurements. The first measurement is used with calibration phase first and the later 2 measurements are using the parameters from that first calibration.

Right temporal - 3 repeat measurements. All 3 measurements are using the parameters from the left measurement first calibration.

Left parietal - 3 repeat measurements. The first measurement is used with calibration phase first and the later 2 measurements are using the parameters from that first calibration.

Right parietal - 3 repeat measurements. All 3 measurements are using the parameters from the left measurement first calibration.

Left occipital - 3 repeat measurements. The first measurement is used with calibration phase first and the later 2 measurements are using the parameters from that first calibration.

Right occipital - 3 repeat measurements. All 3 measurements are using the parameters from the left measurement first calibration.

Once the signals are obtained using the scanning sequence disclosed herein, in at least one embodiment, the signal is processed using algorithm 9 elaborated herein.

For each pair of head locations, there are 3 measurements on the left and right sides. Correspondingly, 6 OD values (3 on each head side) are first calculated. Next, 9 ΔOD values, pairing each left OD with each right OD, are calculated. The largest 3 ΔOD absolute values out of the 9 ΔOD absolute values are then eliminated. An average Δ0D value is then calculated, using the remaining 6 ΔOD original values.

In some embodiments, the average Δ0D value (calculated using the remaining 6 Δ0D original values) are displayed and/or stored in the measurement data file for that head location.

The OD_CrossMax signal processing algorithm may be implemented for subjects that were deemed hematoma positive based on both the original measurement and the following 3 measurements being positive or more than a predetermined threshold (e.g., 0.2). Without wishing to be bound by theory, in such subjects, because all measurements show a positive hematoma, the basic diagnosis of positive/negative should not change, but the used value may be more stable and can be used as initial value for subsequent monitoring of that subject by repeating (a)-(c) at a plurality of time points subsequent to the first time point.

It will be understood that while the example described herein includes 3 repeat measurements, more or less repeat measurements at each location on each side can be performed. Thus, examples may include 4, 5, 6, 7, 8, 9, 10 or even more repeat measurements. Likewise, while the example described herein eliminates the largest 3 ΔOD absolute values out of the 9 ΔOD absolute values, more ΔOD absolute values may be eliminated, in particular in examples where the number of Δ0D values is higher based on a higher number of repeat measurements at each side on each location.

In some embodiments, the measurements of optical density are performed at one, two, three or more different near infrared wavelengths.

Without wishing to be bound by theory, an increase in the average Δ0D value over a period of time may be indicative of an increase in the intensity of the intracranial hemorrhage or an increase in the size of the hematoma over that period of time. Accordingly, the method disclosed herein may be used for assessing whether an immediate triage procedure needs to be performed on the subject or whether a delay in performing the treatment (e.g., caused by delay in evacuation) may be acceptable.

It must be noted that while the scanning sequence disclosed herein involves 3 repeat measurements, the process may include 2, 3, 4, 5, 6, 7, 8, 9, 10 or more repeat measurements. While increasing the number of repeat measurements may provide improved stability, as the number of repeat measurements increases the time needed for each scan also increases. It is, therefore, desirable to balance the improvement in stability of the signal with the amount of time needed for each scan. It was found that the improvement in stability of the signal plateaus after about 3 repeat measurements, and the gains in stability are offset by the increased time requirement after about 3 repeat measurements. Nevertheless, it will be appreciated that higher number of repeat measurements may be performed if the time needed for each scan is reduced by improvements in the scanning methodology or equipment.

In accordance with at least some embodiments the present disclosure, a proposed protocol for using the systems and methods disclosed herein for monitoring a patient may be as follows:

The first measurement by the optical scanner of a patient is considered “Baseline” measurement. A threshold ΔOD is added to the 4 “Baseline” ΔOD values for each head region. A different threshold may be used for hematoma areas and no hematoma areas.

Any subsequent measurement in any of the head regions larger than the Baseline for that area plus the threshold for that area will be considered as “Hematoma Expansion” in that area.

To get an initial sense of what the threshold values may be, prior data was reanalyzed. Hematoma regions and No-Hematoma regions were analyzed separately. Outlier measurements were removed (highest variability 10%). OD_CrossMax algorithm was used for data processing. For each patient the baseline was subtracted from the measurements (the results of OD_CrossMax) and a histogram was plotted for all the changes from baseline. A threshold was selected as the value under which 90% of the histogram area was captured. Similar analysis was done for the prior healthy volunteers’ database.

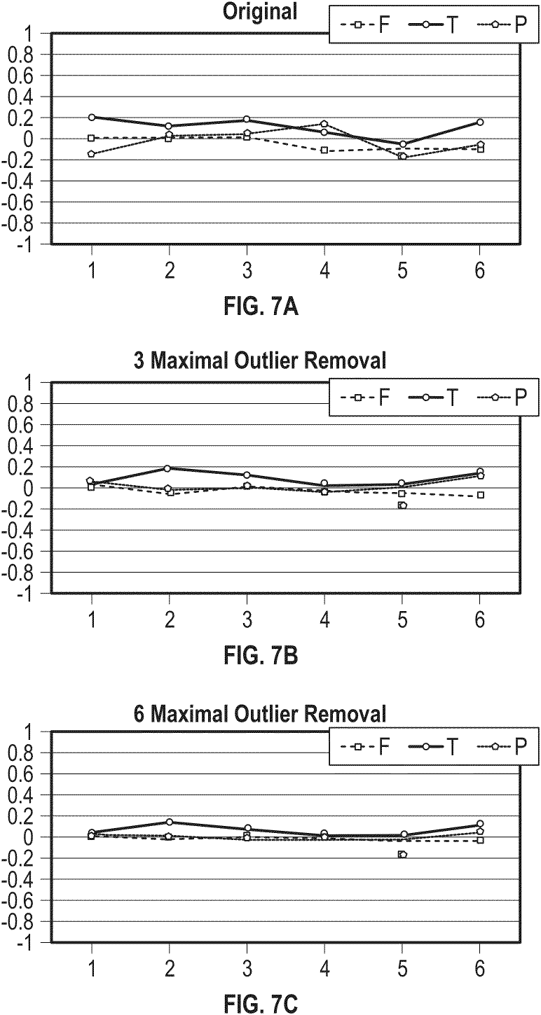

FIGS. 7A-7C show graphs demonstrating the reduction in signal variability and increase in smoothness of the data by increasing the number of outliers eliminated during the data processing.

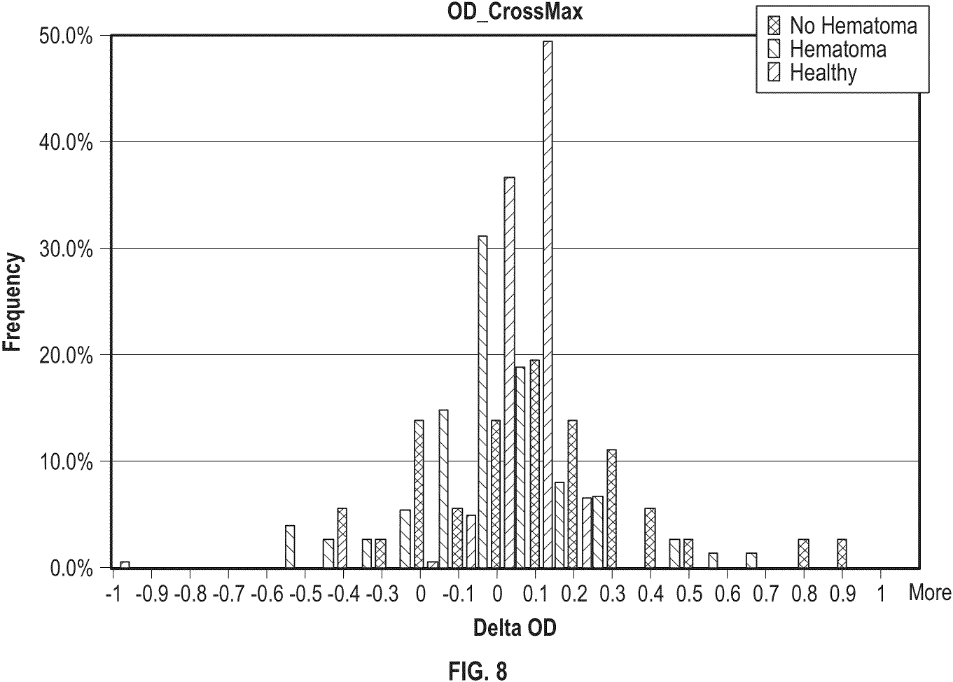

FIG. 8 is a graph showing the distribution of No Hematoma, Hematoma and Healthy volunteers change measurements.

The results of this analysis were:
For the trial subjects with No Hematoma, 90% of the changes from baseline were less than 0.2.
For the trial subjects with Hematoma, 90% of the changes from baseline were less than 0.3.
For healthy volunteer’s cases, 90% of the changes from baseline were less than 0.1.

Based on this analysis, it was apparent that the threshold for detecting expansion for No Hematoma areas, which is influenced by the variability introduced by hair, may be similar to the detection threshold used for the optical scanner (e.g., ΔOD of 0.2).

From trial subjects’ data, it was apparent that the threshold for detecting expansion for Hematoma areas is higher than the No Hematoma areas (e.g., ΔOD of 0.3).

Measurements of healthy volunteers in a controlled environment show higher stability.

Based on the data, 0.2 appears to be a reasonable threshold ΔOD for detecting expansion for No Hematoma areas and 0.3 appears to be a reasonable threshold ΔOD for Hematoma areas.

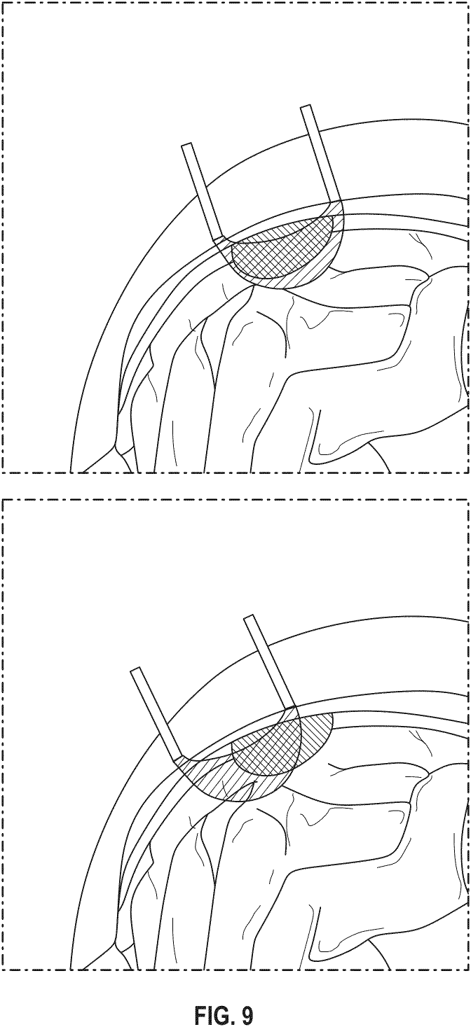

It is believed that there is a reasonable rational why variability in Hematoma areas is higher than in No-Hematoma areas. In No-Hematoma areas the main contributor to variability of the signal is hair trapped under fibers. In Hematoma areas small shifts in measurement location can add to that variability, as illustrated in FIG. 9.

While several exemplary aspects and embodiments have been discussed above, those having skill in the art will recognize certain modifications, permutations, additions and subcombinations that are also within the spirit and scope of this invention.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A method of assessing intracranial hemorrhage in a subject, the method comprising:
(a) at a first time point:
   (aa) measuring, for a plurality of times, optical density (OD) value at first location on a first side of a head of the subject at a first time point, and a first corresponding location on a second side of the head of the subject,
   (ab) computing a set of values of a change (ΔOD) in optical density by subtracting each OD value measured on the first side from each of the OD values measured on the second side,
   (ac) eliminating a predetermined number of largest absolute Δ0D values from the set of Δ0D values, and
   (ad) computing a first average ΔOD value by averaging remaining of Δ0D values from among the set of Δ0D values after step (ac);
(b) at second time point, repeating steps (aa)-(ad) obtain a second average Δ0D value; and
(c) determining a progression of intracranial hemorrhage based on a difference between the first average ΔOD and the second average ΔOD.

Clause 2. The method of clause 1, wherein the predetermined number in step (ac) is in a range from 3 to 6.

Clause 3. The method of clause 1, wherein steps (aa)-(ad) are performed at least three times at each of the first and second time points.

Clause 4. The method of clause 1, wherein the OD values in step (aa) are measured at one or more near-infrared wavelengths.

Clause 5. The method of clause 1, further comprising repeating steps (a)-(c) at a second location on the first side and a second corresponding location on the second side of head of the subj ect.

Clause 6. The method of clause 1, wherein the second time point comprises a plurality of time points.

Clause 7. The method of clause 6, further comprising storing the difference between the first average ΔOD and the second average ΔOD obtained in step (c) for each of the plurality of time points in a non-transitory memory.

Clause 8. The method of clause 1, wherein an increase in absolute value of the difference obtained at step (c) is indicative of an increase in intensity of the intracranial hemorrhage.

Clause 9. The method of clause 1, wherein measuring the optical density value comprises applying an optical probe to the head of the subject.

Clause 10. The method of clause 1, further comprising:
(d) repeating steps (a)-(c) at a plurality of locations on the first side of the head of the subject and corresponding plurality of locations on the second side of the head of the subject;
(e) determining a location of the intracranial hemorrhage based on an average Δ0D value at each of the plurality of locations on the first side and the corresponding plurality of locations on the second side; and
(f) providing a graphical display of the location of the intracranial hemorrhage.

Clause 11. A non-transitory machine-readable medium storing instructions to cause one or more processors to perform operations comprising:
(a) at a first time point:
   (aa) measuring, for a plurality of times, optical density (OD) value at first location on a first side of a head of the subject at a first time point, and a first corresponding location on a second side of the head of the subject,
   (ab) computing a set of values of a change (ΔOD) in optical density by subtracting each OD value measured on the first side from each of the OD values measured on the second side,
   (ac) eliminating a predetermined number of largest absolute Δ0D values from the set of Δ0D values, and
   (ad) computing a first average ΔOD value by averaging remaining of Δ0D values from among the set of Δ0D values after step (iii);
(b) at second time point, repeating steps (aa)-(ad) obtain a second average Δ0D value; and
(c) determining a progression of intracranial hemorrhage based on a difference between the first average ΔOD and the second average ΔOD.

Clause 12. The non-transitory machine-readable medium clause 11, wherein the predetermined number in step (ac) is in a range from 3 to 6.

Clause 13. The non-transitory machine-readable medium of clause 11, wherein steps (aa)-(ad) are performed at least three times at each of the first and second time points.

Clause 14. The non-transitory machine-readable medium of clause 11, wherein the OD values in step (aa) are measured at one or more near-infrared wavelengths.

Clause 15. The non-transitory machine-readable medium of clause 11, wherein the second time point comprises a plurality of time points.

Clause 16. The non-transitory machine-readable medium of clause 15, wherein the operations further comprise storing the difference between the first average ΔOD and the second average ΔOD obtained in step (c) for each of the plurality of time points in a non-transitory memory.

Clause 17. The non-transitory machine-readable medium of clause 11, wherein an increase in absolute value of the difference obtained at step (c) is indicative of an increase in intensity of the intracranial hemorrhage.

Clause 18. The non-transitory machine-readable medium of clause 11, wherein measuring the optical density value comprises applying an optical probe to the head of the subject.

Clause 19. The non-transitory machine-readable medium of clause 11, wherein the operations further comprise:
(d) repeating steps (a)-(c) at a plurality of locations on the first side of the head of the subject and corresponding plurality of locations on the second side of the head of the subject;
(e) determining a location of the intracranial hemorrhage based on an average Δ0D value at each of the plurality of locations on the first side and the corresponding plurality of locations on the second side; and
(f) providing a graphical display of the location of the intracranial hemorrhage.

Clause 20. A system comprising:
an optical probe configured to measure optical density from a portion of a subject’s head;
a memory device to store instructions;
one or more processors operably coupled to the optical probe and configured to execute the instructions stored on the memory device, the instructions to cause the one or more processors to:
   (a) at a first time point:
      (aa) cause the optical probe to measure, for a plurality of times, optical density (OD) value at first location on a first side of a head of the subject at a first time point, and a first corresponding location on a second side of the head of the subject,
      (ab) compute a set of values of a change (ΔOD) in optical density by subtracting each OD value measured on the first side from each of the OD values measured on the second side,
      (ac) eliminate a predetermined number of largest absolute Δ0D values from the set of Δ0D values, and
      (ad) compute a first average ΔOD value by averaging remaining of Δ0D values from among the set of Δ0D values after step (ac);
   (b) at second time point, repeat steps (aa)-(ad) obtain a second average Δ0D value; and
   (c) determine a progression of intracranial hemorrhage based on a difference between the first average ΔOD and the second average ΔOD.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

As used herein, the term “about” preceding a quantity indicates a variance from the quantity. The variance may be caused by manufacturing tolerances or may be based on differences in measurement techniques. The variance may be up to 10% from the listed value in some instances. Those of ordinary skill in the art would appreciate that the variance in a particular quantity may be context dependent and thus, for example, the variance in a dimension at a micro or a nano scale may be different than variance at a meter scale.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1. A method of assessing intracranial hemorrhage in a subject, the method comprising:
(a) at a first time point:
(aa) measuring, for a plurality of times, optical density (OD) value at first location on a first side of a head of the subject at a first time point, and a first corresponding location on a second side of the head of the subject,
(ab) computing a set of values of a change (ΔOD) in optical density by subtracting each OD value measured on the first side from each of the OD values measured on the second side,
(ac) eliminating a predetermined number of largest absolute ΔOD values from the set of ΔOD values, and
(ad) computing a first average ΔOD value by averaging remaining of ΔOD values from among the set of ΔOD values after step (ac);
(b) at second time point, repeating steps (aa)-(ad) obtain a second average ΔOD value; and
(c) determining a progression of intracranial hemorrhage based on a difference between the first average ΔOD and the second average ΔOD.

2. The method of claim 1, wherein the predetermined number in step (ac) is in a range from 3 to 6.

3. The method of claim 1, wherein steps (aa)-(ad) are performed at least three times at each of the first and second time points.

4. The method of claim 1, wherein the OD values in step (aa) are measured at one or more near-infrared wavelengths.

5. The method of claim 1, further comprising repeating steps (a)-(c) at a second location on the first side and a second corresponding location on the second side of the head of the subject.

6. The method of claim 1, wherein the second time point comprises a plurality of time points.

7. The method of claim 6, further comprising storing the difference between the first average ΔOD and the second average ΔOD obtained in step (c) for each of the plurality of time points in a non-transitory memory.

8. The method of claim 1, wherein an increase in absolute value of the difference obtained at step (c) is indicative of an increase in intensity of the intracranial hemorrhage.

9. The method of claim 1, wherein measuring the optical density value comprises applying an optical probe to the head of the subject.

10. The method of claim 1, further comprising:
(d) repeating steps (a)-(c) at a plurality of locations on the first side of the head of the subject and corresponding plurality of locations on the second side of the head of the subject;
(e) determining a location of the intracranial hemorrhage based on an average ΔOD value at each of the plurality of locations on the first side and the corresponding plurality of locations on the second side; and
(f) providing a graphical display of the location of the intracranial hemorrhage.

11. A non-transitory machine-readable medium storing instructions to cause one or more processors to perform operations comprising:
(a) at a first time point:
(aa) measuring, for a plurality of times, optical density (OD) value at first location on a first side of a head of a subject at a first time point, and a first corresponding location on a second side of the head of the subject,
(ab) computing a set of values of a change (ΔOD) in optical density by subtracting each OD value measured on the first side from each of the OD values measured on the second side,
(ac) eliminating a predetermined number of largest absolute ΔOD values from the set of ΔOD values, and
(ad) computing a first average ΔOD value by averaging remaining of ΔOD values from among the set of ΔOD values after step (iii);
(b) at second time point, repeating steps (aa)-(ad) obtain a second average ΔOD value; and
(c) determining a progression of intracranial hemorrhage based on a difference between the first average ΔOD and the second average ΔOD.

12. The non-transitory machine-readable medium claim 11, wherein the predetermined number in step (ac) is in a range from 3 to 6.

13. The non-transitory machine-readable medium of claim 11, wherein steps (aa)-(ad) are performed at least three times at each of the first and second time points.

14. The non-transitory machine-readable medium of claim 11, wherein the OD values in step (aa) are measured at one or more near-infrared wavelengths.

15. The non-transitory machine-readable medium of claim 11, wherein the second time point comprises a plurality of time points.

16. The non-transitory machine-readable medium of claim 15, wherein the operations further comprise storing the difference between the first average ΔOD and the second average ΔOD obtained in step (c) for each of the plurality of time points in a non-transitory memory.

17. The non-transitory machine-readable medium of claim 11, wherein an increase in absolute value of the difference obtained at step (c) is indicative of an increase in intensity of the intracranial hemorrhage.

18. The non-transitory machine-readable medium of claim 11, wherein measuring the optical density value comprises applying an optical probe to the head of the subject.

19. The non-transitory machine-readable medium of claim 11, wherein the operations further comprise:
(d) repeating steps (a)-(c) at a plurality of locations on the first side of the head of the subject and corresponding plurality of locations on the second side of the head of the subject;
(e) determining a location of the intracranial hemorrhage based on an average ΔOD value at each of the plurality of locations on the first side and the corresponding plurality of locations on the second side; and
(f) providing a graphical display of the location of the intracranial hemorrhage.

20. A system comprising:
an optical probe configured to measure optical density from a portion of a subject’s head;
a memory device to store instructions;
one or more processors operably coupled to the optical probe and configured to execute the instructions stored on the memory device, the instructions to cause the one or more processors to:
(a) at a first time point:
(aa) cause the optical probe to measure, for a plurality of times, optical density (OD) value at first location on a first side of a head of the subject at a first time point, and a first corresponding location on a second side of the head of the subject,
(ab) compute a set of values of a change (ΔOD) in optical density by subtracting each OD value measured on the first side from each of the OD values measured on the second side,
(ac) eliminate a predetermined number of largest absolute ΔOD values from the set of ΔOD values, and
(ad) compute a first average ΔOD value by averaging remaining of ΔOD values from among the set of ΔOD values after step (ac);
(b) at second time point, repeat steps (aa)-(ad) obtain a second average ΔOD value; and
(c) determine a progression of intracranial hemorrhage based on a difference between the first average ΔOD and the second average ΔOD.
